(19) United States
(12) Patent Application Publication
(10) Pub. No.: US 2026/0295155 A1
(43) Pub. Date: Oct. 1, 2026

(54) A method of establishing a flow delivery rate of a medicament delivery device

(71) Applicant: SHL Medical AG, Zug (CH)

(72) Inventors: Thomas Daniel James, Baltimore, MD (US); Florence Olivia Stevenson, Baltimore, MD (US); Martin Michael Coyne, III, Boonton, NJ (US); Christopher James Franzese, Boonton, NJ (US)

(21) Appl. No.: 19/480,424

(22) PCT Filed: Apr. 18, 2024
(86) PCT No.: PCT/EP2024/060486

§ 371 Date: Oct. 31, 2025

Related U.S. Application Data
(60) Provisional application No. 63/463,526, filed on May 2, 2023.

(30) Foreign Application Priority Data
Jun. 2, 2023 (EP) 23176902.7

(51) Int. Cl. A61M 5/168; A61M 5/142; A61M 5/44

(52) U.S. Cl. CPC A61M 5/16813; A61M 5/14248; A61M 5/16881; A61M 5/445; A61M 2005/14252; A61M 2205/3368; A61M 2205/364; A61M 2205/366; A61M 2205/50; A61M 2205/8212

(57) ABSTRACT

The present disclosure provides a method of establishing a flow delivery rate of a medicament delivery device. The medicament delivery device comprises a body containing a medicament, a delivery member for delivering the contained medicament to a user, and a tube extending between the body and the delivery member. The tube is configured to deliver the contained medicament from the body to the delivery member. The method comprising the following steps in the following order: (i) obtaining time information containing the amount of the time needed for the medicament to be warmed up to a target temperature, (ii) obtaining the information of the volume of the tube, and (iii) establishing a flow delivery rate that is greater than zero and lower than the volume divided by the amount of the time; wherein the contained medicament within the body is configured to be delivered to the tube with the established flow delivery rate.

### TECHNICAL FIELD

The present disclosure generally relates to a method of establishing a flow delivery rate of a medicament delivery device, and particularly to a method of establishing a flow delivery rate for a medicament delivery device comprising a tube.

### BACKGROUND

Medicament delivery devices such as auto-injectors are generally known for the self-administration of a medicament by patients without formal medical training. For administrating high volume and/or viscosity medicament, or multiple different medicaments, a motor-driven medicament delivery device is desirable.

Many injectable drug products, such as biologics, require cold storage to ensure active pharmaceutical ingredient (API) stability, primarily to protect protein integrity. Biologics are kept in cold storage throughout manufacture, and the time out of the temperature range (e.g., above cold temperatures) is carefully monitored throughout the process of manufacture, transport, and ultimate patient distribution. Secondarily, cold environments reduce the likelihood of microbial growth after a potential microbial ingress event (such as during compounding). Thus, even drug products that do not require refrigeration for API stability may require refrigeration after being dispensed to the patient for microbial stability.

For drugs and drug-device combination products provisioned for administration in the home, either by the patient, a lay caregiver, or an in-home health care professional, this can greatly complicate the administration workflow. Upon receipt, such medications must still be kept in cold storage and are often stored in a home or dedicated refrigerator. Before administration, these medications must then be removed from the fridge for defined “warm-up” periods.

Warm-up periods serve two main purposes. First, cold medications are uncomfortable to inject and therefore undesirable for patients. Second, cold medications have mechanical effects that can be problematic for drug delivery as the viscosity of the medicament increased when the medicament is at a low temperature. Many drugs configured for subcutaneous administration, particularly biologics, contain high concentrations of drug product, which inherently increase the viscosity of the drug product. The relationship between protein concentration and viscosity is not perfectly understood, particularly considering that many of these drug products exhibit significant non-Newtonian behaviours. Typically, however, this relationship is exponential. Small reductions in temperature can therefore produce significant increases in medication viscosity. This phenomenon can be prohibitive for drug delivery devices, as they may not be able to produce forces sufficient to overcome flow resistance or doing so would create unacceptable downstream consequences (e.g., cracked primary containers, exorbitant power requirements).

As such, it is common practice for the drug product manufacturer to advise users to take their medication out of the refrigerator for a period of time before the injection time, to allow the drug product to equilibrate to room temperature. However, this does not solve the usability problem entirely; users who are time-constrained may outright ignore the guidance, or worse, skip the dose if they are unable to allow adequate time to warm the drug. In extreme cases, there have been reports of patients microwaving injectable products to accelerate warming, which can damage the product or in some cases, cause severe burns. And if the user does not receive adequate training or does not consult the IFU, they may go ahead and inject the drug cold for lack of knowledge to the contrary, causing the use considerations above to become relevant.

Last, some drug products have narrow stability and/or potency windows, so while the user is instructed to allow 30 minutes for the drug to warm, that duration must not exceed 45 minutes, should the dose be rendered compromised.

There is clearly a demonstrated need on the basis of patient safety and drug product efficacy for more effective means of the thermal management of drug products when injected in the home setting.

### SUMMARY

The invention is defined by the appended claims, to which reference should now be made.

There is hence provided a method of establishing a flow delivery rate of a medicament delivery device; wherein the medicament delivery device comprises a body containing a medicament, a delivery member for delivering the contained medicament to a user, and a tube extending between the body and the delivery member, the tube is configured to deliver the contained medicament from the body to the delivery member, the method comprising the following steps in the following order:
obtaining time information containing the amount of time (T) needed for the medicament to be warmed up to a target temperature;
obtaining the information of the volume of the tube (V); and
establishing a flow delivery rate within a range calculated by the following equation:

$$0 < Q < \frac{V}{T}$$

where T is the obtained amount of time; V is the volume of the tube; and Q is the range of the flow delivery rate;
wherein the contained medicament within the body is configured to be delivered to the tube with the established flow delivery rate.

The method is configured to establish a flow delivery rate for delivering the contained medicament within the body to the tube that can make the medicament stay in the tube long enough to be warmed up to the target temperature. Therefore, the operation steps of using the medicament delivery device can be simplified, e.g., the user doesn't need to perform a step of leaving the medicament container in the room temperature environment, e.g., outside of a refrigerator, for a period of time.

Preferably, according to another embodiment, the amount of time is greater than 10 seconds.

Preferably, according to another embodiment, the step of obtaining time information, the step comprises the following steps in the following order:
obtaining information about at least one of a characteristic of the contained medicament, the temperature of the contained medicament, the ambient temperature of the tube, and a characteristic of the tube; and
calculating the time information based on the obtained information; or
searching in a database with the obtained information to fetch the time information matching with the obtained information.

Preferably, according to another embodiment, the method comprises the following steps in the following order:
obtaining the temperature information of the medicament within the tube;
comparing the obtained temperature information and the target temperature;
sending a stop signal for stopping the delivery of the contained medicament within the body to the tube when the obtained temperature information is not reached the target temperature.

Preferably, according to another embodiment, the step of obtaining the temperature information of the medicament within the tube comprises the step of:
obtaining the temperature information of the medicament within the tube at the end of the tube that is connected to the delivery member.

Preferably, according to another embodiment, the step of obtaining time information containing the amount of the time needed for the medicament to be warmed up to a target temperature comprises a step of obtaining time information containing the amount of the time needed for the medicament to be warmed up to the ambient temperature of the tube.

According to another aspect of the invention, a pump of a medicament delivery device is provided.

The medicament delivery device comprises a body containing a medicament, a delivery member for delivering the contained medicament to a user, and a tube extending between the body and the delivery member, the tube is configured to deliver the contained medicament from the body to the delivery member.

Preferably, according to another embodiment, the pump comprises a processor; and a driver electrically connected to the processor and operably connected to the body of the medicament delivery device; wherein the processor is configured to control the driver to force the medicament contained within the body into the tube with a flow delivery rate that is established by the method as mentioned above and/or to stop the drive from forcing the medicament contained within the body into the tube. Preferably, according to another embodiment, the processor is configured to perform the method as mentioned above.

Preferably, according to another embodiment, the driver comprises a pneumatic pressure power source or a hydraulic pressure power source.

Preferably, according to another embodiment, the driver is configured to pressurize the medicament contained within the body into the tube with the established flow delivery rate.

Preferably, according to another embodiment, the pump of the medicament delivery device comprises a processor electrically connected to the driver, and an electrical power source connected to the processor. The processor and the electrical power source are accommodated within the reusable body.

Preferably, according to another embodiment, the pneumatic power source is configured to output gas to the body of the medicament delivery device and expel the medicament contained within the body by increasing the gas pressure around the medicament container. The hydraulic power source is configured to output liquid to the body of the medicament delivery device and expel the medicament contained within the body by increasing the liquid pressure around the medicament container. When the pressure power source is the pneumatic power source, the body comprises a fluid-tight chamber that is gas-tight. When the fluid pressure power source is hydraulic, the body comprises a fluid-tight chamber that can be either gas-tight or liquid-tight.

Preferably, according to another embodiment, the driver comprises a piezo pump configured to cause fluid to output from the fluid pressure power source. Furthermore, the piezo pump is preferably used as the pneumatic power source. Using a piezo pump can be advantageous as it can provide quiet or silent operation and can provide lower power operation when compared to motor-driven solutions.

Preferably, according to another embodiment, the driver comprises a motor-based fluid pump configured to cause fluid to output from the fluid pressure power source.

Preferably, according to another embodiment, when the driver comprises the pneumatic power source, the pneumatic power source comprises a pressurized gas canister.

Preferably, according to another embodiment, when the driver comprises the pneumatic power source, the pneumatic power source is a well-controlled diaphragm pump when lower cost or complexity is desired.

Preferably, according to another embodiment, when the driver comprises the pneumatic power source, the pneumatic power source comprises an electronics engine, e.g., a MEMS engine, and a liquid substance. In this example, the engine is configured to trigger an electrochemical reaction of the liquid substance to generate propellant gas.

Preferably, according to another embodiment, the driver is a pneumatic power source. In this embodiment, the fluid outputted from the fluid pressure power source is gas, e.g., air or nitrogen.

Preferably, according to another embodiment, the driver is a hydraulic power source. In this embodiment, the fluid outputted from the fluid pressure power source is liquid, e.g., water or oil.

Preferably, according to another embodiment, the user interface is electrically connected to the processor.

Preferably, according to another embodiment, the user interface is a button protruding from the outer surface of the reusable body.

Preferably, according to another embodiment, the user interface is a screen or touch panel arranged on the outer surface of the reusable body.

According to another aspect of the invention, a tube of a medicament delivery device is provided.

The medicament delivery device comprises a body containing a medicament, and a delivery member for delivering the contained medicament to a user; wherein the tube is configured to extend between the body and the delivery member, the tube is configured to deliver the contained medicament from the body to the delivery member

Preferably, according to another embodiment, the tube comprises a source end configured to be fluidly connected to the body of the medicament delivery device; a delivery end configured to be fluidly connected to the delivery member of the medicament delivery device.

Preferably, according to another embodiment, a tube body extends between the source end and the delivery end.

Preferably, according to another embodiment, the tube further comprises a heat-exchanging element attached to the tube body.

Alternatively, or preferably, according to another embodiment, a length of the tube body extending from the delivery end is wound in a serpentine or spiral pattern.

Alternatively, or preferably, according to another embodiment, the tube body comprises a thermally conductive additive.

Preferably, according to another embodiment, the tube is made of a cyclic olefin copolymer or ethylene vinyl acetate.

Preferably, according to another embodiment, the tube comprises a tube valve.

Preferably, according to another embodiment, the tube valve is an in-line valve included in the delivery tube.

Preferably, according to another embodiment, the tube valve of the tube is a one-way valve so that the fluid cannot flow towards the medicament container through the delivery tube.

Preferably, according to another embodiment, the tube valve of the delivery tube is an umbrella valve or a Belleville valve or a ball valve or a pinch valve.

Preferably, according to another embodiment, when the tube further comprises a heat-exchanging element that is attached to the tube body.

Preferably, according to another embodiment, the heat-exchanging element is a finned heat exchanger.

Preferably, according to another embodiment, the heat-exchanging element is annular.

According to another aspect of the invention, a sub-assembly of a medicament delivery device is provided.

The medicament delivery device comprises a body containing a medicament, a delivery member for delivering the contained medicament to a user, and a tube extending between the body and the delivery member, the tube is configured to deliver the contained medicament from the body to the delivery member.

Preferably, according to another embodiment, the sub-assembly comprises the body of the medicament delivery device and a pump as mentioned above.

Preferably, according to another embodiment, the body comprises a thermal sensor configured to detect the temperature of the contained medicament.

According to another aspect of the invention, a medicament delivery device is provided.

Preferably, according to another embodiment, the medicament delivery device comprises a delivery member for delivering the contained medicament to a user, a tube as mentioned above connected to the delivery member, and the tube being configured to deliver the contained medicament from the body to the delivery member.

Preferably, according to another embodiment, the dimension of the delivery member and the dimension of the tube is set with the following equation:

$$\frac{{L}_{d}}{{R}_{d}^{4}} > \frac{{L}_{t}}{{R}_{t}^{4}}$$

where L_{d} is the length of the delivery member; R_{d} is the inner diameter of the delivery member; Lₜ is the length of the tube; and Rₜ is the inner diameter of the tube.

Preferably, according to another embodiment, the medicament delivery device comprises comprising: a temperature sensor configured to detect the temperature of the medicament within the tube at the delivery end of the tube.

Preferably, according to another embodiment, the medicament delivery device comprises a sub-assembly as mentioned above,

Preferably, according to another embodiment, the medicament delivery device comprises a medicament container containing a medicament.

Preferably, according to another embodiment, the medicament container comprises a flexible bag.

Preferably, according to another embodiment, the medicament delivery device is portable.

Preferably, according to another embodiment, the medicament delivery device is a body worn, underneath or over the clothing.

Preferably, according to another embodiment, the medicament delivery device is an injection device, e.g., an infusion device or an on-body injector.

Preferably, according to another embodiment, the delivery member is an injection needle or an insertion needle with a soft cannula.

Preferably, according to another embodiment, the medicament delivery device comprises a reusable body and a changeable medicament delivery member.

Preferably, according to another embodiment, the medicament delivery device comprises a wireless communication receiver connected to the processor.

Preferably, according to another embodiment, the medicament delivery device comprises a wireless communication transmitter connected to the processor.

Preferably, according to another embodiment, the wireless communication receiver is configured to receive a wireless signal from a remote device or an information tag.

Preferably, according to another embodiment, the wireless communication transmitter is configured to transmit a wireless signal to a remote device or an information tag.

The medicament delivery devices described herein can be used for the treatment and/or prophylaxis of one or more of many different types of disorders. Exemplary disorders include but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a medicament delivery device configured to deliver contained medicament with a tube.

FIG. 2 shows a relationship between flow rate, outlet temperature, and tube length with water that flows through the tube.

FIG. 3 shows a relationship between viscosity and temperature. Segur and Oberstar, Viscosity of Glycerol and Its Aqueous Solutions, 1949. The viscosity of an aqueous glycerol solution as a function of temperature. The glycerol ratio by weight is 70%, yielding a nominal viscosity of 22.5 cP at a temperature of 20° C., which is a similar viscosity to many concentrated medicaments.

FIG. 4 shows a method of the invention for establishing a flow delivery rate of a medicament delivery device.

### DETAILED DESCRIPTION

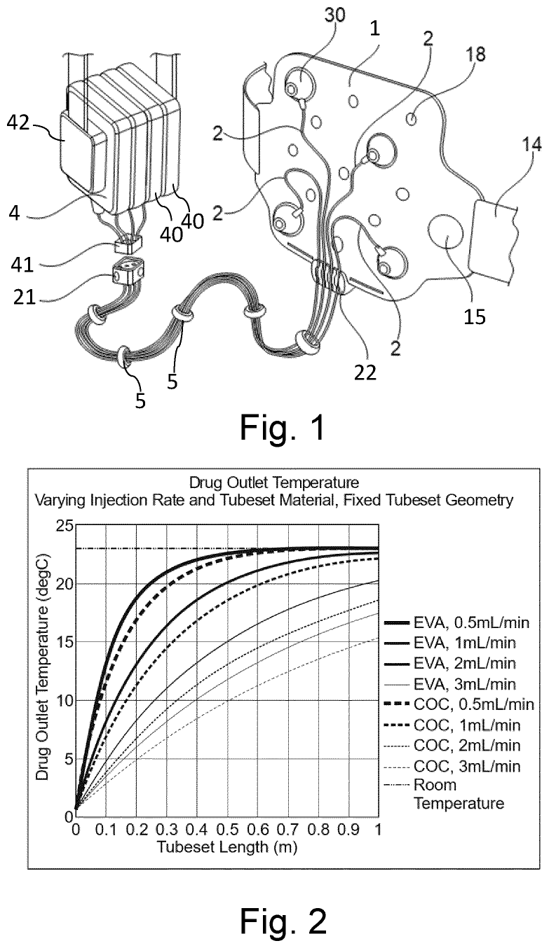

A method 500 of establishing a flow delivery rate of a medicament delivery device and the medicament delivery device using the established flow delivery rate is disclosed in the text below. The medicament delivery device comprises a body 4 containing a medicament, a delivery member 30 for delivering the contained medicament to a user, and a tube 2 extending between the body 4 and the delivery member 30, the tube 2 is configured to deliver the contained medicament from the body 4 to the delivery member 30, as shown in FIG. 1. In a preferred example, the medicament delivery device is configured for large-volume injection, e.g., 10 ml to 200 ml injection, preferably, 50 ml to 100 ml injection. It should be noted that the medicament delivery device is also capable of performing the small-volume injection, e.g., 1 ml to 5 ml, or below 1 ml. In a preferred example, the medicament delivery device comprises multiple tubes 2 and multiple delivery members connected to the multiple tubes respectively, as shown in FIG. 1. In a preferred example, the medicament delivery device can be used with a wearable pad 1 comprising multiple apertures 18 and a wearable feature 14, e.g., a stripe or clip, for being worn by the user. The multiple apertures 18 are configured to guide the user to place the delivery members at the correct delivery sites. In a preferred example, the multiple tubes 2 are collected with a clip 22 attached to the pad. In a preferred example, the tubes 2 are collected to a connection hub 21 configured to connect to the body 4 of the medicament delivery device. In one example, the body 4 comprises a counter hub 41 configured to be attached to the connection hub 21. Alternatively, the medicament delivery device may only comprise one single tube connected to multiple delivery members. In a preferred example, the delivery member is an injection needle. Alternatively, the delivery member is a soft cannula configured to be embedded under the skin of the user. For example, the soft cannular can be embedded under the skin of the user by the medical professional, or the medicament delivery device might comprise a needle inserter having an introducing needle positioned within the soft cannular, the introducing needle is configured to penetrate the skin of the user and retracted from the soft cannular after penetration. In a preferred example, the medicament is contained within a medicament container within the body 4 of the medicament delivery device. In a preferred example, the medicament container is a syringe, a cartridge, or an elastic bag. In a preferred example, the body 4 of the medicament delivery device comprises multiple medicament containers. In one example, the body 4 comprises multiple sections 40 to accommodate multiple medicament containers respectively. In a preferred example, the medicament delivery device is an injection device.

The medicament delivery device comprises a pump configured to provide a power source operably acting on the medicament such that the medicament can be pumped into the tube and expelled from the delivery member to the user. The pump is configured to be controlled to pump the contained medicament within the body 4 to the tube 2 with a flow delivery rate established by the method 500 as shown in FIG. 4.

The method 500 comprises three steps 501, 502, and 503 in the following order:
obtaining time information containing the amount of time (T) needed for the medicament to be warmed up to a target temperature;
obtaining the information of the volume (V) of the tube; and
establishing a flow delivery rate within a range calculated by the following equation:

$$0 < Q < \frac{V}{T}$$

where T is the obtained amount of time; V is the volume of the tube; and Q is the established flow delivery rate;
wherein the contained medicament within the body is configured to be delivered to the tube with the established flow delivery rate.

The target temperature is the temperature at that the medicament in this temperature is suitable to be delivered to the user of the medicament delivery device.

The method is configured to ensure the medicament thermal equilibration relative to the ambient temperature of the tube 2 (i.e., room temperature) before medicament delivery via geometric and flow constraints. Thus, the maximum target temperature is the ambient temperature of the tube. As the established flow delivery rate is configured to fill up the volume of the tube in a period of time that the medicament can reach the target temperature of the tube 2 via the thermal equilibration, the user can start to use the medicament delivery device immediately without a pre-administration warm-up step, e.g., placing the medicament in the room temperature for a period of time to wait for the medicament to be warmed up to room temperature. Furthermore, as the volume of the tube 2 is much less than the medicament container, the time needed for the medicament to be warmed up is shorter when only the medicament in the tube, e.g., 0.5 ml or fewer, that needs to be warmed up comparing to the time needed for the entire contained medicament, e.g., 100 ml, to be warm up.

Furthermore, in a preferred example, the step of obtaining time information containing the amount of time needed for the medicament to be warmed up to a target temperature comprises a step of obtaining time information containing the amount of the time needed for the medicament to be warmed up to the ambient temperature of the tube. As the target temperature should be close to the ambient temperature, e.g., room temperature, thus, even if the medicament is a temperature that is slightly lower than the room temperature might be still suitable to be delivered to the user, using the ambient temperature of the tube to set the amount of time for calculating the flow delivery rate can make sure that the medicament that is delivered with the established flow delivery rate is warmed closer to the room temperature, thus, the user can have a better medicament delivery experience, e.g., less painful. Furthermore, using the ambient temperature of the tube to set the amount of time for calculating the flow delivery rate can also simplify the method. As the target temperature might be different for different medicaments, thus, the user might need to input different medicament information every time before using the medicament delivery device. On the other hand, by using the ambient temperature of the tube to set the amount of time for calculating the flow delivery rate, the amount of time can be calculated based on the ambient temperature of the tube and the initial temperature of the medicament without the information about the types of medicaments.

Preferably, the medicaments that are needed to be warmed up are configured to be kept in the refrigerator, thus, the medicaments are usually warmed up from above 0 degrees Celsius to the ambient temperature of the tube. Thus, the target temperature is greater than the temperature within the refrigerator, e.g., above 0-4 degrees Celsius and is lower or equal to the ambient temperature of the tube.

In one example, the time information is predetermined. For example, as the greatest thermal equilibration effect is presented when the medicament is furthest from room temperature, the most significant temperature change occurs within the first 10 seconds of the medicament delivery. Thus, in a preferred example, the amount of time is greater than 10 seconds. For example, when the volume of the tube is 1 ml, the flow delivery rate is preferred to be greater than 0 ml/s and lower than 0.1 ml/s. In a preferred example, the amount of time is about 30, seconds as in many foreseeable medicament delivery systems, the time to reach the steady state room temperature is about 30 seconds due to convective heat transfer. The example where the time information is predetermined is suitable for those medicaments that are less temperature sensitive.

Alternatively, the time information can be calculated based on the conductive and convective heat transfer equations, with the information about the heat capacity of the materials of the tube and/or the medicament to be delivered. In this example, the step of obtaining time information comprises a step of obtaining information about at least one of characteristics of the contained medicament, the temperature of the contained medicament, the ambient temperature of the tube, and a characteristic of the tube. For example, the heat capacity of the materials of the tube and/or the medicament, the length of the tube, the temperature around the tube, and the temperature of the medicament can be obtained. After the information is obtained, the time information can be calculated based on the obtained information or the time information can be fetched from a database by matching the obtained information.

For example, as shown in FIG. 2, when the tubes have different lengths and/or are made of different materials, the flow delivery rate for the contained liquid (in particular, at the outlet of the tube) to be warmed up from 0 degree Celsius to a common room temperature (23 degrees Celsius in the example shown in FIG. 2) can be tested and grouped in the database. For example, FIG. 2 shows a test result with water flowing through different tubes with a flow delivery rate that can have the water at the outlet of the tube warmed up to room temperature. Thus, the heat exchange rate can be calculated for tubes with different lengths and made of a different materials.

In one example where the medicament delivery devices are identical, from the same manufacturer or same hospital/pharmacy, for example, the time information can be fetched with the information of the medicament. Alternatively, when the medicament is identical, but the medicament delivery device is different, the user gets the medicament delivery device from another hospital, for example, the time information can be fetched with the information of the material of the tube and the length of the tube.

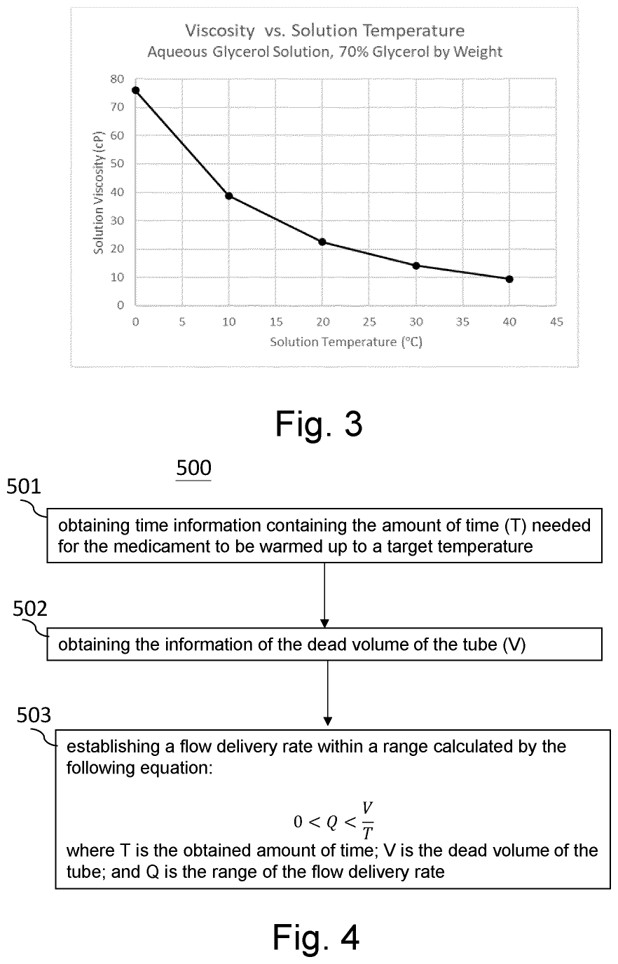

The medicament delivery device that is configured to deliver the medicament with the flow delivery rate established by the method mentioned above uses the walls of the tube as a means of heat transfer from the ambient environment into the medicament over the duration of its procession through the tube, such that the medicament has significantly warmed, and thus, reduced its viscosity (as shown in FIG. 3), and thus, reduced the required motive force to flow at a given flow delivery rate, before encountering the majority of the fluid path's restriction, e.g., flowing into the delivery member.

Furthermore, in a preferred example, the method further comprises a step of sending a stop signal for stopping the delivery of the contained medicament within the body to the tube if the medicament not yet reaches the target temperature. In this example, the medicament delivery device comprises multiple sensors to detect the temperature of the temperature information of the medicament within the tube and the ambient temperature of the tube. In a preferred example, the temperature information of the medicament within the tube is detected at the end of the tube that is connected to the delivery member.

In a preferred example, a pump 42 of the medicament delivery device as mentioned above is provided. The pump 42 comprises a processor and a driver electrically connected to the processor and operably connected to the body 4 of the medicament delivery device. The processor is configured to control the driver to force the medicament contained within the body into the tube with a flow delivery rate that is established by the method mentioned above and/or to stop the driver from forcing the medicament contained within the body into the tube. In one example, the pump is built-in into the body of the medicament delivery device.

Alternatively, the pump can be releasably attached to the body of the medicament delivery device.

The driver can be a pneumatic pressure power source, a hydraulic pressure power source, a peristaltic pump, a motor with a motor-driven shaft, or a motor-driven gear set. In one example, the processor is configured to receive a signal from an external device. For example, the method mentioned above is performed in a smartphone or a cloud server, and the established flow delivery rate and/or the stop signal is sent to the processor of the pump to control the driver. In this example, the cost of the processor can be reduced. Alternatively, the processor is configured to perform the method mentioned above. In this example, the medicament delivery device can be used in an environment with low electromagnetic radiation, for those patients who cannot be exposed to much electromagnetic radiation, for example.

In a preferred example, a tube 2 of a medicament delivery device as mentioned above is provided. The tube 2 is generally made of plastic and the tube 2 is elastic. The tube 2 is configured to extend between the body 4 of the medicament delivery device and the delivery member of the medicament delivery device. The tube 2 being configured to deliver the contained medicament from the body to the delivery member. In one example, the tube 2 is configured to be connected to the body 4 of the medicament delivery device by the user. For example, the tube 2 is connected to the body 4 of the medicament delivery device via a connection hub 21 as shown in FIG. 1. The tube 2 comprises a source end configured to be fluidly connected to the body of the medicament delivery device; a delivery end configured to be fluidly connected to the delivery member of the medicament delivery device; and a tube body extending between the source end and the delivery end. In one example, the tube 2 comprises a heat-exchanging element 5 attached to the tube body. Additionally or alternatively, a length of the tube body extending from the delivery end is wound in a serpentine or spiral pattern.

The heat-exchanging element is configured to improve the capacity of the tube for convective heat transfer. The heat-exchanging element can be an annular or a finned heat exchanger.

Additionally or alternatively, an additive is included in the material of the tube to substantially improve its thermal conductivity and thus its heat transfer capability.

In one example where the length of the tube body extending from the delivery end is wound in a serpentine or spiral pattern, the user can be instructed to place the tube body, in particular, the serpentine or spiral pattern portion on the skin. Thus, the heat transfer between the skin and the tube can be maximized. In a preferred example, the serpentine or spiral pattern portion of the tube body is adjacent to the delivery end of the tube.

In one example, the tube is made of a cyclic olefin copolymer or ethylene vinyl acetate.

In a preferred example, a sub-assembly of the medicament delivery device as mentioned above is provided. The sub-assembly comprises the body of the medicament delivery device and the pump as mentioned above. In a preferred example, the body comprises a thermal sensor configured to detect the temperature of the contained medicament.

Furthermore, the medicament delivery device as mentioned above comprises the tube 2 and the delivery member 30. In a preferred example, the dimension of the delivery member and the dimension of the tube is set with the following equation:

$$\frac{{L}_{d}}{{R}_{d}^{4}} > \frac{{L}_{t}}{{R}_{t}^{4}}$$

where L_{d} is the length of the delivery member; R_{d} is the inner diameter of the delivery member; Lₜ is the length of the tube; and R^{t} is the inner diameter of the tube.

The force to balance the hydraulic restriction across a cylindrical object is calculated by Hagen-Poiseuille equation, meaning when the flow delivery rate is consistent, the force to move the liquid within the cylindrical object is proportional to the dimension of the cylindrical object

$$\frac{L}{{R}^{4}}$$

(the ratio between the length and the inner diameter of the cylindrical object) times the dynamic viscosity of the liquid.

As mentioned above, the viscosity of the medicament is generally decreases when the temperature of the medicament is increased, as shown in FIG. 3. Thus, when the flow delivery rate is consistent, the tube 2 and the delivery member 30 that are dimensioned to fulfil the relation as described by the equation above makes sure the delivery member provides a greater hydraulic restriction than the tube. As a result, the pump of the medicament delivery device can be used in the most efficient way as the output force from the pump can be set by calculating the diameter of the delivery member without a need to increase the output force for pushing the medicament through the tube. In other words, with the dimensions relation between the tube and the delivery member as mentioned above, as long as the pump is able to force the medicament through the delivery member, the pump is capable of forcing the medicament through the tube. Furthermore, as the output force is set based on the dimension of the delivery member and the target dynamic viscosity (meaning that the medicament is warmed up to the target temperature), if the medicament that arrives at the delivery member is not warm enough, the hydraulic restriction will be high due to the high dynamic viscosity; as a result, the medicament might not be expelled as the predetermined output force is not high enough to move the medicament with high viscosity through the delivery member. Thus, the user will only receive the medicament when the medicament is warmed up to the target temperature.

In another example, the medicament delivery device comprises a temperature sensor configured to detect the temperature of the medicament within the tube at the delivery end of the tube. Furthermore, in another example, the medicament delivery device comprises the sub-assembly as mentioned above.

The inventive concept has mainly been described above with reference to a few examples. However, as is readily appreciated by a person skilled in the art, other embodiments than the ones disclosed above are equally possible within the scope of the inventive concept, as defined by the appended claims.

1-17. (canceled)

18. A method of establishing a flow delivery rate of a medicament delivery device, wherein the medicament delivery device comprises a body containing a medicament, a delivery member for delivering the contained medicament to a user, and a tube extending between the body and the delivery member, the tube is configured to deliver the contained medicament from the body to the delivery member, the method comprising the following steps in the following order:
obtaining time information containing the amount of time (T) needed for the medicament to be warmed up to a target temperature;
obtaining the information of the volume of the tube (V); and
establishing a flow delivery rate within a range calculated by the following equation:
$$0 < Q < \frac{V}{T}$$
where T is the obtained amount of time; V is the volume of the tube; and Q is the range of the flow delivery rate,
wherein the contained medicament within the body is configured to be delivered to the tube with the established flow delivery rate.

19. The method according to claim 18, wherein the amount of time is greater than 10 seconds.

20. The method according to claim 18, wherein the step of obtaining time information, the step comprises the following steps in the following order:
obtaining information about at least one of a characteristic of the contained medicament, the temperature of the contained medicament, the ambient temperature of the tube, and a characteristic of the tube; and
calculating the time information based on the obtained information; or
searching in a database with the obtained information to fetch the time information matching with the obtained information.

21. The method according to claim 18, comprising the following steps in the following order:
obtaining the temperature information of the medicament within the tube;
comparing the obtained temperature information and the target temperature; and
sending a stop signal for stopping the delivery of the contained medicament within the body to the tube when the obtained temperature information is not reached the target temperature.

22. The method according to claim 21, wherein the step of obtaining the temperature information of the medicament within the tube comprises the step of:
obtaining the temperature information of the medicament within the tube at the end of the tube that is connected to the delivery member.

23. The method according to claim 18, wherein the step of obtaining time information containing the amount of the time (T) needed for the medicament to be warmed up to a target temperature comprises a step of obtaining time information containing the amount of the time (T) needed for the medicament to be warmed up to the ambient temperature of the tube.

24. A pump of a medicament delivery device; wherein the medicament delivery device comprises a body containing a medicament, a delivery member for delivering the contained medicament to a user, and a tube extending between the body and the delivery member, the tube is configured to deliver the contained medicament from the body to the delivery member; the pump comprising:
a processor; and
a driver electrically connected to the processor and operably connected to the body of the medicament delivery device,
wherein the processor is configured to control the driver to force the medicament contained within the body into the tube with a flow delivery rate that is established by the method according to claim 18 and/or to stop the drive from forcing the medicament contained within the body into the tube.

25. The pump according to claim 24, wherein the processor is configured to perform the method according to claim 18.

26. The pump according to claim 24, wherein the driver comprises a pneumatic power source configured to pressurize the medicament contained within the body into the tube with the established flow delivery rate.

27. A tube of a medicament delivery device, wherein the medicament delivery device comprises a body containing a medicament, a delivery member for delivering the contained medicament to a user; wherein the tube is configured to extend between the body and the delivery member, the tube is configured to deliver the contained medicament from the body to the delivery member; the tube comprising:
a source end configured to be fluidly connected to the body of the medicament delivery device;
a delivery end configured to be fluidly connected to the delivery member of the medicament delivery device; and
a tube body extending between the source end and the delivery end,
wherein the tube further comprises a heat-exchanging element attached to the tube body, and/or
wherein a length of the tube body extending from the delivery end is wound in a serpentine or spiral pattern, and/or
wherein the tube body comprises a thermally conductive additive.

28. The tube according to claim 27, wherein the tube is made of a cyclic olefin copolymer or ethylene vinyl acetate.

29. The tube according to claim 27, wherein when the tube further comprises a heat-exchanging element that is attached to the tube body, wherein the heat-exchanging element is a finned heat exchanger.

30. The tube according to claim 27, when the tube further comprises a heat-exchanging element attached to the tube body, wherein the heat-exchanging element is annular.

31. A sub-assembly of a medicament delivery device, wherein the medicament delivery device comprises a body containing a medicament, a delivery member for delivering the contained medicament to a user, and a tube extending between the body and the delivery member, the tube is configured to deliver the contained medicament from the body to the delivery member, the sub-assembly comprising:
the body of the medicament delivery device; and
a pump according to claim 24,
wherein the body comprises a thermal sensor configured to detect the temperature of the contained medicament.

32. A medicament delivery device comprising:
a delivery member for delivering the contained medicament to a user; and
a tube according to claim 27 connected to the delivery member, the tube is configured to deliver the contained medicament from the body to the delivery member,
wherein the dimension of the delivery member and the dimension of the tube is set with the following equation:
$$\frac{{L}_{d}}{{R}_{d}^{4}} > \frac{{L}_{t}}{{R}_{t}^{4}}$$
where L_{d} is the length of the delivery member; R_{d} is the inner diameter of the delivery member; Lₜ is the length of the tube; and Rₜ is the inner diameter of the tube.

33. The medicament delivery device according to claim 32, comprising: a temperature sensor configured to detect the temperature of the medicament within the tube at the delivery end of the tube.

34. The medicament delivery device according to claim 32, wherein the medicament delivery device comprises the sub-assembly according to claim 31.
